(19) United States
(12) Patent Application Publication
(10) Pub. No.: US 2025/0273315 A1
(43) Pub. Date: Aug. 28, 2025

(54) System Method and Computer Program Product for Improving Care of Patients with Chronic Cancer

(71) Applicant: Celloscope Ltd., Tel Aviv (IL)

(72) Inventors: Yuval NAVEH, Haifa (IL); Tamir ALOUSH, Kiryat Ono (IL); Levi TEITZ, Tel Aviv (IL); Shahar DAVIDSON, Rehovot (IL); Tomer SHUSSMAN, Kfar Saba (IL); Hila GLICK, Ramat Hasharon (IL); Itamar BEN DAVID, Giv'atayim (IL); Guy KEREM, Kiryat Ono (IL)

(21) Appl. No.: 19/061,254

(22) Filed: Feb. 24, 2025

Related U.S. Application Data
(63) Continuation-in-part of application No. 18/939,288, filed on Nov. 6, 2024.
(63) Continuation-in-part of application No. 18/975,890, filed on Dec. 10, 2024.
(63) Continuation-in-part of application No. 19/049,400, filed on Feb. 10, 2025.
(63) Continuation-in-part of application No. 19/051,712, filed on Feb. 12, 2025.
(63) Continuation-in-part of application No. 19/056,378, filed on Feb. 18, 2025.
(60) Provisional application No. 63/596,479, filed on Nov. 6, 2023.
(60) Provisional application No. 63/612,587, filed on Dec. 20, 2023.
(60) Provisional application No. 63/557,740, filed on Feb. 26, 2024.
(60) Provisional application No. 63/557,747, filed on Feb. 26, 2024.
(60) Provisional application No. 63/557,753, filed on Feb. 26, 2024.
(60) Provisional application No. 63/557,757, filed on Feb. 26, 2024.
(60) Provisional application No. 63/557,762, filed on Feb. 26, 2024.

(51) Int. Cl. G16H 20/10; A61B 5/00; A61B 5/11; G16H 20/40

(52) U.S. Cl. CPC G16H 20/10; A61B 5/112; A61B 5/4839; A61B 5/4848; A61B 5/6898; G16H 20/40; A61B 2562/0219

(57) ABSTRACT

System and method for treating diseased patients, the method comprising, for at least one diseased patient who bears a mobile phone having a built-in IMU and to whom treatment is being administered according to a treatment plan, repeatedly deriving gait parameters from raw data generated by the IMU including identifying at least one change/s in at least one gait parameter/s over time; and/or using the gait parameters, as repeatedly derived, to monitor the patient typically including making at least one change in the treatment plan which may be based on at least the changes in the gait parameters.

### CROSS-REFERENCE TO RELATED APPLICATIONS

The present application is a continuation-in-part of U.S. application Ser. No. 18/939,288, filed Nov. 6, 2024, and of U.S. application Ser. No. 18/975,890, filed Dec. 10, 2024, and of U.S. application Ser. No. 19/049,400, filed Feb. 10, 2025, and of U.S. application Ser. No. 19/051,712 entitled “System, Method, and Computer Program Product for Tracking Cognitive Status Based On Repeated Mobility Assessments” filed Feb. 12, 2025, and of U.S. patent application Ser. No. 19/056,378 filed Feb. 18, 2025, the entire contents of each of which being hereby fully incorporated herein by reference. The present application further claims benefit, directly or indirectly, of the following provisional applications, the entire contents of each of which being fully incorporated herein by reference: Application No. 63/557,740, filed Feb. 26, 2024 and entitled “Mobility Assessment in Visually Impaired Individuals”; Application No. 63/557,747, filed Feb. 26, 2024; Application No. 63/557,753, filed Feb. 26, 2024; Application No. 63/557,757, filed Feb. 26, 2024, Application No. 63/557,762, filed Feb. 26, 2024; Application No. 63/596,479, filed Nov. 6, 2023; and Application No. 63/612,587, filed Dec. 20, 2023.

### FIELD OF THIS DISCLOSURE

The present invention relates generally to computerized analysis of motion, and more particularly to computerized analysis of human motion which receives sensor outputs borne by a human, typically in real time.

### BACKGROUND FOR THIS DISCLOSURE

Research on cancer and gait is described in:
ascopubs.org/doi/abs/10.1200/OP.2024.20.10_suppl.427
and in:
ashpublications.org/blood/article/134/4/374/260685/Gait-speed-grip-strength-and-clinical-outcomes-in.

Mobilise-D was a project which set out to set standards by generating “tools that can detect and measure how well someone walks, including speed, symmetry/efficiency, pain, and endurance”, denigrating “Existing mobility endpoints based on performance, patient self-reporting, and one-off assessment” as “resource intensive and [lacking in] sensitivity”. Mobilise-D IMI 2019-2024-Mobilise-D

Other modern research in the field is described in:
mobilise-d.eu/
pmc.ncbi.nlm.nih.gov/articles/PMC7371223/
www.nature.com/articles/s41746-021-00513-5

OneStep is an FDA-listed medical app, downloadable from GooglePlay, that uses smartphone motion sensors to provide immediate, clinically-validated feedback on gait, inter alia.

The disclosures of all publications and patent documents mentioned above and elsewhere in the specification, and of the publications and patent documents cited therein directly or indirectly, are hereby incorporated herein by reference in their entirety. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

Certain embodiments provide a machine learning process to estimate standard assessment.

Certain embodiments herein are configured for using the feature vector or the time-series as described herein to learn PRO scores e.g. “PRO questionnaires on physical function (PF) and fatigue (PROMIS Fatigue 7a & PROMIS Physical Function 10a)”.

Certain embodiments herein are configured for combining various gait and mobility measures from ongoing measurements using machine learning methods, thus learning a composite measure that is correlated with a given standard assessment endpoint\outcome measure.

At least the following embodiments are provided:

Embodiment 1. A method for treating diseased patients, the method comprising (for at least one diseased patient who bears a mobile phone having an imu (built-in inertial measurement unit) and to whom treatment is being administered according to a treatment plan), repeatedly deriving gait parameters from raw data generated by the imu including identifying at least one change/s in at least one gait parameter/s over time; and/or using the gait parameters, as repeatedly derived, to monitor the patient typically including making at least one change in the treatment plan which may be based on at least the changes in the gait parameters.

Embodiment 2. A method according to any preceding embodiment, wherein the change in the treatment plan comprises transitioning to a new treatment protocol.

Embodiment 3. A method according to any preceding embodiment, wherein the change in the treatment plan comprises terminating a treatment protocol.

Embodiment 4. A method according to any preceding embodiment, wherein the treatment plan administers a dosage of a medical agent, and wherein the change in the treatment plan comprises changing the dosage.

Embodiment 5. A method according to any preceding embodiment, wherein the medical agent comprises a drug.

Embodiment 6. A method according to any preceding embodiment, wherein the medical agent comprises radiation.

Embodiment 7. A method according to any preceding embodiment wherein the gait parameters are used to identify that the diseased patient is suffering from a known motor side effect of a treatment protocol being used and accordingly making said change in the treatment plan.

Embodiment 8. A method according to any preceding embodiment wherein first and second treatment protocols are known to be characterized by first and second motor side effects, and wherein at least one gait parameter useful for recognizing the first motor side effect is computed in patients undergoing the first treatment protocol and typically not in at least one patient not undergoing the first treatment protocol, and wherein gait parameters useful for recognizing the second motor side effect are computed in patients undergoing the second treatment protocol and typically not in at least one patient not undergoing the second treatment protocol.

Embodiment 9. A method according to any preceding embodiment wherein the treatment plan comprises a cancer treatment plan being administered to a cancer patient.

Embodiment 10. A system comprising at least one hardware processor configured to carry out a method for treating patients with disease, the method comprising: repeatedly deriving, for at least one diseased patient who bears a mobile phone having an imu (built-in inertial measurement unit) and to whom treatment is being administered according to a treatment plan, gait parameters from raw data generated by the imu thereby to identify changes in the gait parameters over time; and/or using the gait parameters, as repeatedly derived, to monitor the patient including making at least one change in the treatment plan based on at least the changes in the gait parameters.

Embodiment 11. A computer program product, comprising a non-transitory tangible computer readable medium having computer readable program code embodied therein, said computer readable program code adapted to be executed to implement a method for treating patients with disease, the method comprising, for at least one diseased patient who bears a mobile phone having an imu (built-in inertial measurement unit) and to whom treatment is being administered according to a treatment plan: repeatedly deriving gait parameters from raw data generated by the imu thereby to identify changes in the gait parameters over time; and/or using the gait parameters, as repeatedly derived, to monitor the patient including making at least one change in the treatment plan based on at least the changes in the gait parameters.

Also provided, excluding signals, is a computer program comprising computer program code means for performing any of the methods shown and described herein when said program is run on at least one computer; and a computer program product, comprising a typically non-transitory computer-usable or readable medium e.g. non-transitory computer-usable or readable storage medium, typically tangible, having a computer readable program code embodied therein, said computer readable program code adapted to be executed to implement any or all of the methods shown and described herein. The operations in accordance with the teachings herein may be performed by at least one computer specially constructed for the desired purposes, or a general-purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display, e.g., on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g., BLE) or wired (e.g., USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g., via a wired or cellular telephone network, or a computer network such as the Internet.

The apparatus of the present invention may include, according to certain embodiments of the invention, machine-readable memory containing or otherwise storing, a program of instructions, which, when executed by the machine, implements all or any subset of the apparatus, methods, features, and functionalities of the invention shown and described herein. Alternatively, or in addition, the apparatus of the present invention may include, according to certain embodiments of the invention, a program as above which may be written in any conventional programming language, and optionally a machine for executing the program, such as but not limited to a general-purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities, e.g., within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing systems, communication devices, processors (e.g., digital signal processors (DSPs), microcontrollers, field programmable gate arrays (FPGAs), application specific integrated circuits (ASICs), etc.) and other electronic computing devices. Any reference to a computer, controller, or processor, is intended to include one or more hardware devices, e.g., chips, which may be co-located or remote from one another. Any controller or processor may, for example, comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs), or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g., a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface, or other system illustrated or described herein. Any suitable computerized data storage, e.g., computer memory, may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein, which may, for example, include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus, the term user interface or “UI” as used herein includes also the underlying logic which controls the data presented to the user, e.g., by the system display, and receives and processes and/or provides to other modules herein, data entered by a user, e.g., using her or his workstation/device.

### BRIEF DESCRIPTION OF THE DRAWINGS

Certain embodiments of the present invention are illustrated in the following drawings; in the block diagrams, arrows between modules may be implemented as APIs, and any suitable technology may be used for interconnecting functional components or modules illustrated herein in a suitable sequence or order, e.g., via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML. According to one embodiment, one of the modules may share a secure API with another module. Communication between modules may comply with any customized protocol or customized query language, or may comply with any conventional query language or protocol.

FIGS. 1a-1b, 2 and 3 are simplified flowchart illustrations of methods (in which all or any subset of the operations may be performed) which are useful for performing embodiments of the present invention.

Methods and systems included in the scope of the present invention may include any subset or all of the functional blocks shown in the specifically illustrated implementations by way of example, in any suitable order, e.g., as shown. Flows may include all or any subset of the illustrated operations, suitably ordered, e.g., as shown. Tables herein may include all or any subset of the fields and/or records and/or cells and/or rows and/or columns described.

Computational, functional or logical components described and illustrated herein can be implemented in various forms, for example as hardware circuits, such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines, and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device, and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if, and as appropriate, and which may employ any suitable architectural option, such as but not limited to FPGA, ASIC, or DSP, or any suitable combination thereof.

Any hardware component mentioned herein may in fact include either one or more hardware devices, e.g., chips, which may be co-located or remote from one another.

Any method described herein is intended to include, within the scope of the embodiments of the present invention, also any software or computer program performing all or any subset of the method's operations, including a mobile application, platform or operating system, e.g., as stored in a medium, as well as combining the computer program with a hardware device to perform all or any subset of the operations of the method.

Data can be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary, or off-line; which is of any type or level or amount or category of volatility, differentiation, mutability, accessibility, addressability, capacity, performance and energy use; and which is based on any suitable technologies such as semiconductor, magnetic, optical, paper, and others.

### DETAILED DESCRIPTION OF CERTAIN EMBODIMENTS

It is appreciated that there are known indicators for cancer, but chronic oncology patients are better treated at home. The clinics may benefit if they were able to receive a remote indication of the status of patients undergoing chemo at home according to embodiments of this invention.

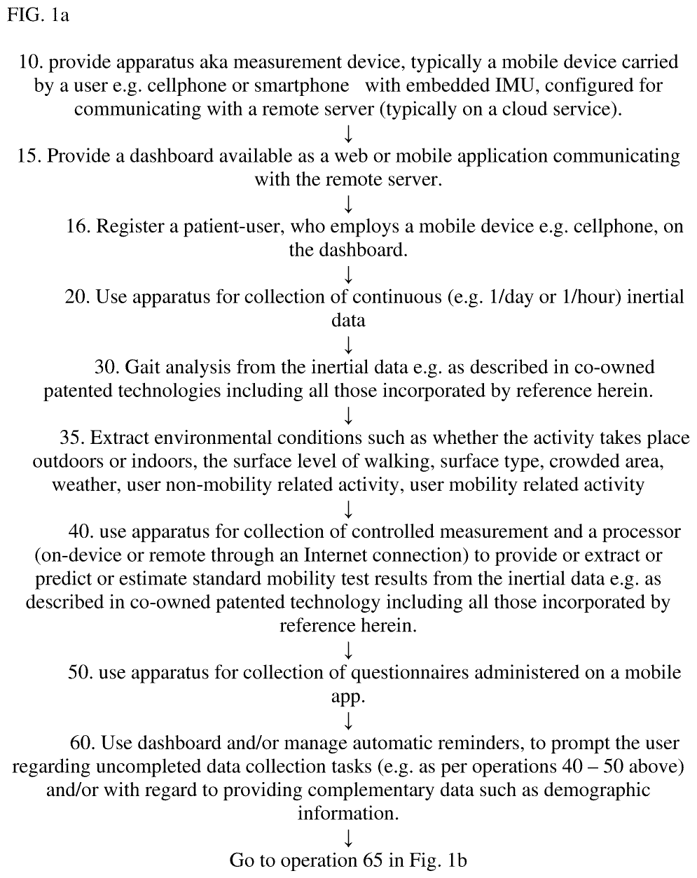

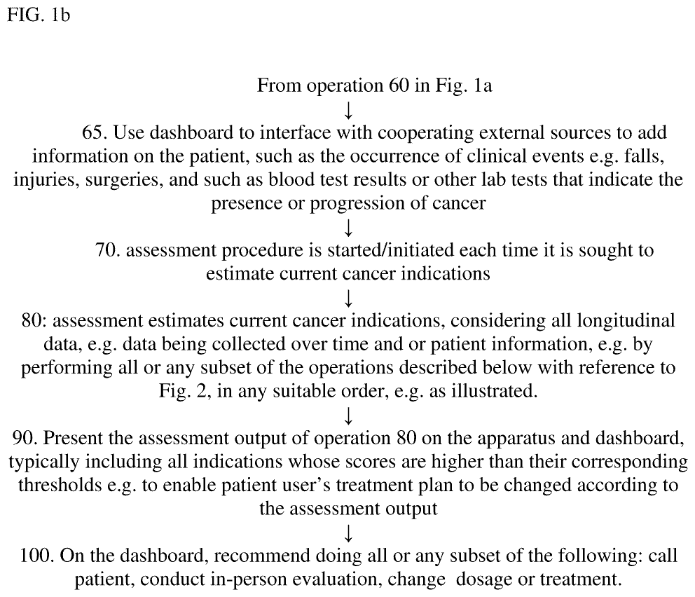

A method for monitoring patients with chronic cancer is now described in detail with reference to FIGS. 1a-1b, taken together. All or any subset of the following operations 10, 15, 16, . . . may be provided in any suitable order, e.g. as follows:

Operation 10. provide apparatus aka measurement device, typically a mobile device carried by a user e.g. cellphone or smartphone with embedded IMU, configured for communicating with a remote server (typically on a cloud service).

Operation 15. Provide a dashboard available as a web or mobile application communicating with the remote server.

Operation 16. Register a patient-user, who employs a mobile device e.g. cellphone, on the dashboard. Typically, registration includes entering the user's cellphone number and/or patient demographics and treatment information, such as prescribed medicine and blood tests measuring substances (PSA-prostate-specific antigen or CA-125) at registration.

Operation 20. Use apparatus for collection of continuous (or frequent, say 1/day or 1/hour) inertial data calibrated to (say) the north, and (if the user's cellphone provides these functionalities) any measurements of available sensors, such as but not limited to GPS locations, altimeter, barometer, microphone, humidity sensor, and a light sensor over time.

Operation 30. Gait analysis-use a processor (on-device or remote through an Internet connection) to extract gait analysis from the inertial data as described in co-owned U.S. patent application Ser. No. 16/659,832 (US Publication US 2020/0289027), Ser. No. 17/500,744 (US Publication US 2022/0111257), and/or US 2023/0137198 which claims priority from U.S. Ser. No. 63/272,839 (the disclosures of which are hereby incorporated by reference herewithin in their entirety).

Gait analysis in operation 30 may include all or any subset of the following:
30-1. Spatiotemporal analysis: evaluation of gait properties such as gait cycle time, cadence, gait speed, stride length, right and left step length, base width, right and left single support and stance time and percentage, double support time and percentage, e.g. as described in co-owned US 2020/0289027 A1 (the disclosure of which is hereby incorporated by reference herewithin in its entirety).
30-2. Kinematics: identifying all or any subset of lower body joint angles over the gait cycle, e.g. as described in co-owned U.S. Ser. No. 17/666,180 published as US20230137198A1 (the disclosure of which is hereby incorporated by reference herewithin in its entirety).
30-3. Kinetic: evaluation of gait properties related to torques and forces such as ground forces and center of feet pressure over the gait cycle, and the analysis of center of pressure's sway over the gait cycle.
30-4. Variability: expressed using any suitable operationalization and or definition, e.g. as described herein, as well as definitions as defined herein, may represent deviation of any of the gait properties above between different strides over the same walk. A walk may, for example, include a one-minute time window which usually includes 20-70 strides. It may also be longer, and may measure a walk starting when more than 10 strides takes place, and ending when there are no strides for more than a minute. Variability may be processed in two forms, first aggregated, e.g., over the entire walk, e.g., as the standard deviation of each gait property's value, and/or second—as a sequence of differences in gait property value/s over time. Differences are typically between consecutive strides, and standard deviation is typically an aggregated value over the number of strides, whereas a sequence of differences provides a difference value for each pair of consecutive strides in the sequence of strides (except for the last or the first one).
35. Extract environmental conditions-use a processor (on-device or remote through Internet connection) to extract environmental conditions such as whether the activity takes place outdoors or indoors, the surface level of walking, surface type, crowded area, weather, user non-mobility related activity, user mobility related activity, e.g. as described in detail below.

Any suitable technique may be employed to extract environmental conditions from background or passive walks, such as but limited to all or any subset of the following:
1. Outdoor vs. Indoor—One approach is that a segment of walking is considered outdoor walking if it has more than 20 consecutive strides. Any other walk segment containing stops or turns is considered indoor walking. It does not depend on whether the user walks outdoors or indoors. Another approach is adding/using the surrounding noise captured via the microphone to determine whether the user is walking outdoors (www.sciencedirect.com/science/article/pii/S2405959515300795).
2. Surface level-use altimeter data to measure the change in altitude over time, measure the altitude difference of each stride, and compute the average inclination per stride. Use stride lengths from layer 30A described herein to compute the inclination per meter.

For this purpose, the repetitive imu pattern and method for “Reconstruction of the Pattern Representation” e.g. as described in: co-owned US 2022/0111257 entitled “ . . . physical rehabilitation” (the disclosure of which is hereby incorporated by reference herewithin in its entirety), may be employed. Specifically, segmentation of a repetitive pattern may be provided, which typically comprises one or both of the following two stages:

Stage 1: Sampling pattern candidates, each of which typically comprises a data segment. It is appreciated that it may not be known a priori, which candidate data segments actually exhibit the repetitive pattern, hence are “successful candidates”. For example, it may be desired to identify a gait cycle which is the repetitive pattern which a patient is walking. However, some data segments (IMU recordings over an interval of time e.g.) may be “unsuccessful candidates” e.g. because they turn out to have been recorded when the patient was, say, sitting or standing, rather than ambulating e.g. walking. Also, the length of a gait cycle (pattern duration), for various subjects, may vary from 0.5 seconds to, say, 4 seconds. Thus, a segment of a given length such as 2 seconds, may represent one subject's entire gait cycle, half of a cycle for another subject, and 2 or 3 cycles for a third, fast moving patient.

Typically, the repetitive pattern is a segment (e.g. sequence) of the original data which occurs plural times. Thus, finding the pattern by sampling is doable. Sampling of candidates may comprise taking data segments with a fixed duration every x seconds. In order to reflect a significant pattern, the candidates may have a long enough duration. For example, a 2-second duration may be used, sampling candidates every 4 seconds.

Stage 2: Scoring the candidates with a score which reflects the objective of this process which is finding a pattern that describes the data well e.g. a pattern which, if repeated, at the indexes of repetitions, this reconstructs the original data accurately e.g. with minimal or below threshold Euclidean distance between the original data and the reconstructed data. Therefore, the better and more accurately the candidate covers the data, the more preferable that candidate is. For instance, the correlation of the candidate with the data may be computed over time over each of the channels. The total correlation over time may be the mean correlation, or some other central tendency, of all of the channels' correlations. Peaks of the total correlation over time are detected with standard signal processing methods, typically configured to filter in only peaks with the highest values over a sliding window of fixed duration. Peaks with high correlation values imply a repetition of the pattern, however the repetition may be shorter than the duration of the pattern, since correlated data intervals may overlap with one another. Hence, the real repetition is typically either the data interval that matches the pattern duration, or the interval between correlation matches (peaks with high values of correlation), typically the shorter of the two.

Eventually, the score may be computed as a sum of the repetition durations multiplied by their correlation values which may be regarded as reflecting the measure of coverage that the pattern candidate captures. A candidate may be dismissed if no sequential repetitions are detected, indicating that this pattern is not repetitive. Parameters configured for this process may include: peaks' sliding window duration parameter which may be determined by the shortest repetition expected; e.g. the parameter may be 0.4 seconds as capturing a significant pattern with higher frequency than this value enables is not expected. Other parameters configured for this process may include the correlation threshold to consider a peak as a potential repetition, and/or the minimal number of consecutive repetitions, and/or the maximum deviation of the intervals' durations between consecutive repetitions. Example parameters' values may be: correlation threshold: 0.5, minimal number of consecutive repetitions: 3, maximum deviation of the intervals' durations between consecutive repetitions: 40% of deviation from the average duration.

Once the candidate with the highest score has been identified, all or any subset of the following stages may be carried out:

1. Refinement of the pattern—the segments between consecutive repetitions have the correct length of the repetitive pattern; moreover, their average pattern may be shown to achieve a higher average correlation with all the repetitions. So the average of the segments between repetitions may get a higher score as a candidate, and therefore, may be deemed the “true” repetitive pattern. All segments may be cut to fit the shortest interval between repetitions, to avoid overlaps.

2. Detection of the repetitions-find peaks of the total correlation between the final pattern and the original data, e.g. as was done when evaluating the scores of the pattern candidates; the same configured parameters as before may be used again. Only intervals between consecutive peaks as repetitions may be considered; although this might miss, say, the first and the last repetitions of a sequence or sequences of two repetitions, this ensures that the considered intervals are real instances (repetitions of) the repetitive pattern. In addition, the first and the last repetitions in a sequence might be a bit different than the rest of the repetitions. Thus, typically, precise detection of real repetitions may be prioritized over “covering” all repetitions, or over the goal of detecting every single repetition.

Reconstruction of the Pattern Representation may include representation of the measurement in a reference frame which may be defined as follows: the X-axis is the course of the movement of the IMU (of the end-user wearing the IMU e.g.), the Y-axis is, say, toward the sky, the Z-axis is, say, toward the right (the normal of the X-Y plane). The following operations yield a representation of the repetitions which typically captures all relevant information of the motion being measured while being invariant to irrelevant attributes of the measurement and the analysis, including the base orientation at which the device was set during the measurement, and phase 0 at which the repetitions define the starting point of the repetitive pattern. Once the following 3 stages have been performed, their output typically comprises a standard representation of the pattern, including 3 spatial channels, 3 orientational channels, and a channel for each extra information channel of the original data, with over 100 uniform units dividing the duration of the repetitions.

Stage a—Adjustment and Standardization of Repetitions: For each of the data segments of the repetitions, perform all or any subset of the following operations, suitably ordered e.g. as shown:

a-1. Ignoring the offset of the yaw channel—The north may be irrelevant to defining the frame of reference as defined above, and similarly any other offset of the yaw, since given a motion record of someone walking, the direction of the walk is not relevant, and it is desirable for the representation of that activity to be invariant of the direction. Hence, the average yaw may be subtracted for each repetition, whether the data is calibrated to the north, or showing a false reference of the north. Other techniques may also be used, such as splitting the yaw channel into two—one channel for the azimuth (a smoothed version of the yaw represents the macro changes of the yaw), and the other channel for subtraction of the azimuth from the yaw, which represents the micro changes of the yaw.

a-2. Although at this stage the directions of the X- and Z-axis are not known, the Y-axis (the vertical direction) is known from the orientation channel of the data. Applying (e.g. using conventional rotation arithmetic) the rotations included in the orientation channel, the acceleration channel takes these rotations from the reference frame of the measurement device and embeds these rotations in an “uncalibrated” reference frame, e.g. a reference frame in which the X or the Z axes are arbitrary; they may be found as described in stage B below. Multiplying the rotations of the orientation channel with their inverse mean of orientation on the right produces rotations that are relative to the mean orientation of the segment, aka the relative rotations. By rotating the acceleration channel and producing the relative rotations, the relation between the data and the orientation of the measurement system over the body is eliminated, and what remains are accelerations and relative rotations in the uncalibrated reference frame.

a-3. Displacement and orientation adjustment may be performed. Typically, the data is presented as displacement, although the data could also be presented as accelerations. The displacement is smoother, enables understanding of the motion better for human eyes, and preserves the information of the original data, which may be reversed by derivating the displacement. The reference frame moves with the movement, meaning that the displacement over the data segment may be assumed to be 0 which determines the initial velocity (the velocity may also be assumed not to change over the data segment, meaning that there are no accelerations between repetition, which turns out to be a reasonable assumption for long continuous repetitions. In addition, the effect on the data is minor, which is subtraction of the average acceleration over the data segment (the average acceleration may be kept). This assumption yields benefits: the data may be fixed due to noise or biases of the sensors which are reasonable, and/or displacement is not only closed (as it starts and ends at the same displacement) but also smooth, since the velocity is the same at the beginning and at the end. This makes any shift of data from the end of the segment to its beginning yield the same displacement, yielding invariance to the determination of phase 0, the starting point of the repetitions' segments. The orientation of the starting point and the last point may be constrained to be the same.

Thus, according to certain embodiments, the average acceleration is subtracted from the accelerations and integrated t over the segment to get velocities; then the average velocity is subtracted from the velocities and integrated over the segment to get the displacement. Orientations are handled after the reference frame has been fixed.

a-4. Interpolation e.g. of the displacement and orientation of each segment to have (say) 100 equal time units, to ensure a standard structure for each repetition.

Stage b—Determination of the Course of Movement

The reference frame may be rotated such that X and Z axes will be calibrated, where the course of movement is the X-axis. This rotation depends on the orientation of the measurement system referred to the motion itself; typically, all of the repetitions' representations at this stage are embedded in coherent reference frames, which typically need to be calibrated with the same rotation. This rotation may be estimated using any suitable technique, such as applying principal component analysis (PCA) on the projections of either the displacements or the orientations on the horizontal plane (which is known, since the vertical axis is known). The most significant component of the displacement may imply the direction of the movement because this is the direction in which the most significant changes typically occur. Similarly, the most significant component of the orientations represented as rotation vectors may imply the Z-axis around which the most significant changes of orientations typically occur (e.g. in walking measured from the thigh (e.g. if the IMU is adjacent to the end-user's thigh), the most significant component of the orientations over the horizontal plane is the hip flexion-extension axis). Either of these (the most significant component of the displacement, or the most significant changes of orientations) may be used to rotate the reference frame. Eventually, this yields all of the data represented in a calibrated frame of reference. The orientations may then be converted to Euler representation in the order of Z-X-Y, and the average angle velocities of each orientation channel may be subtracted to verify each of them starts and ends at the same angle, which yields invariance to the initial point of the segmentations e.g. as described above.

Stage c aggregation repetitions. Typically, the repetitions' representations are similar to each other, even initially, and, typically, after standardization of their structure and elimination of irrelevant attributes of measurement and analysis, the repetitions' representations become even more similar. Some tasks may use one aggregated representation of the motion, rather than on each of the repetitions, for example, recognition of the activity being performed by the subject and the position of the measurement device as well. For this kind of task, the aggregated form of the repetitions' representations may be employed. This process may, for example, comprise naive averaging each of the channels over every one of 100 (say) time-units or by dynamic time warping (DTW) techniques. In practice, the naive aggregation method, while less sophisticated, nonetheless works well.

The representation above is useful inter alia for activity recognition (e.g. walking downhill, uphill at various possible slopes) where given a representation of repetitive motion—the activity performed by the subject and/or the position of the sensor e.g. IMU on the body is/are classified, e.g. as the activity was performed and measured.

Typically the above pattern reconstruction/activity classification is applied only to gait cycle intervals/strides/linear gait intervals.

Any suitable method may be employed to obtain labels of the activity classes between which the system seeks to distinguish. The system may match the IMU data with the corresponding altimeter readings (e.g. readings and IMU data having the same time-stamp, are matched) and get the average change in height. The system may use, say, a minute of imu recording and may, accordingly, compute the change of height per second and learn the following relationship: input: 1-minute imu, output: height change (meter/sec). Alternatively or in addition the system may match a repetitive pattern representation with, say, an average change of height per stride, and then learn the following relationship: input: repetitive imu representation, output: height change (meter/stride). Alternatively, users may be prompted to measure each of various types of activities within the app, such as ascending/descending stairs, cycling, running, which yields labels for these types of activity. This may be used as training data when training activity recognition classifiers to predict or identify or recognize the type of activity a user is engaged in, when data is collected passively or when labels are missing.

The term “passive” e.g. passive collection of data is intended to include indications of gait deterioration identified by gait analysis of passively measured imu data, where “passive” refers to imu data measurement which does not require user cooperation each time the imu data is measured; the user may be unaware that imu data is being measured at that time.

Alternatively or in addition, “recognized activity” e.g. as described in co-owned U.S. patent application Ser. No. 16/659,832 (US Publication US 2020/0289027), Ser. No. 17/500,744 (US Publication US 2022/0111257), or US 2023/0137198, the disclosures of which are hereby incorporated by reference in their entirety, may be trained to specific gait activities such as walking downhill (5, 10, 15 degrees) or uphill (5, 10, 15 degrees).

Thus a variation on layer 30a may be provided which unlike layer 30a may be trained for classifying into, say, the following 6 classes: walking downhill (5, 10, 15 degrees) and uphill (5, 10, 15 degrees).

Still referring to extraction of environmental conditions from background or passive walks:
3. Surface type-use accessibility map may be provided; mapping of accessibility level may be performed e.g. in accordance with any teaching in co-owned U.S. Ser. No. 18/975,890 entitled “Pedestrian Navigation Based on Inertial Gait Analysis and GPS Data” (the disclosure of which is hereby incorporated by reference herewithin in its entirety). Map layers may be used to derive terrain type.

Alternatively or in addition, “recognized activity” e.g. as described in co-owned U.S. patent application Ser. No. 16/659,832 (US Publication US 2020/0289027), Ser. No. 17/500,744 (US Publication US 2022/0111257), US 2023/0137198 claiming priority from US 2023/0137198 which claims priority from U.S. Ser. No. 63/272,839 (the disclosures of which are hereby incorporated by reference herewithin in their entirety), may be trained to specific gait activities, such as walking on various different surfaces such as sand or grit or asphalt or grass.
4. Crowded area—The microphone captures the surrounding noise to determine if the user is walking in a crowded area (ris.utwente.nl/ws/portalfiles/portal/220586486/Wang2020sound_based.pdf).
5. Weather—use time and location from GPS to get historical weather data (openweathermap.org/history)
6. User's non-mobility related activity may be used to give two possible examples.
6a. User-smartphone activities such as listening to music, interacting with the phone, and talking over the phone (all of which may be determined using the smartphone OS standard APIs)
6b. The user is talking (not over the phone)—using the sound captured via the microphone to determine whether the user is speaking (docs.nvidia.com/nemo-framework/user-guide/latest/nemotoolkit/asr/speaker_recognition/results.html #speaker-verification-inference). Verification requires a baseline speaker recording which takes place at the patient onboarding. The system then takes the baseline and current audio recordings, and uses the reference method to verify that the patient is speaking.
7. User's mobility-related activity—a data layer which may be similar to “data layer 30A” referred to elsewhere herein, as described in co-owned U.S. patent application Ser. Nos. 16/659,832, 17/500,744, and US 2023/0137198 claiming priority from U.S. Ser. No. 63/272,839 (the disclosures of which are hereby incorporated by reference herewithin in their entirety), may be trained to any specific gait activities such as climbing stairs, jogging, running, and any other form of specific gait activity.

Regarding “recognized activity”, it is appreciated that a cellphone or smartphone with built-in IMU which may include all or any subset of accelerometer, gyroscope, magnetometer, altimeter, and/or barometer, may be used for collection of typically time-stamped, typically continuous inertial data (typically calibrated to the north) and typically time-stamped, typically continuous GPS locations. A hardware processor (on-device or remote through Internet connection) may then be used to extract gait analysis from the inertial data; this motion analysis aka gait analysis may yield (inter alia) a data layer termed herein “Data layer 30a”. This data layer typically includes motion semantic segmentation over time including partitioning motion into intervals which each belong to one of plural categories of recognized activity, e.g., all or any subset of the following five categories:
a. Device Transition—change of the position of the measurement device e.g. phone with accelerometer (the terms “accelerometer” and “IMU” may be interchanged within the present disclosure).
b. Shake—significant movement which is unrecognized (e.g. not falling into any other class); this is characteristic e.g. of a cellphone deployed in a moving vehicle. Typically, if a user moves his phone while he is turning, the class is SHAKE rather than TURN, because the net motion is not that of a turn, and if a user moves her phone while she is standing in place, the class is not STOP but SHAKE, because the net motion is not that of a stop.
c. Stops—the measurement device is stationary, possibly indicating that the subject is standing in place or that the measurement device has been set aside and is not presently on the user's body. This may be identified as velocity zero, however it may be difficult to estimate velocity accurately, directly from IMUs. Alternatively or in addition, if for a period of time accelerations are static or very low e.g. less than 0.05 m/sec**2, this may be used to indicate that the sensor is static, or does not accelerate.
d. Turns—the subject is turning, or changing course, or changing heading, or changing direction of movement. Typically, gyro readings may be used to yield a relative change in heading from one stride to another; a heading change between adjacent strides may be regarded as significant if it is over a threshold (e.g. more than 30 degrees).
e. Strides—detection of linear gait activity. Many repetitive mobility activity may be considered as gait, such as walking, running, climbing stairs, going up/downhill, cycling e.g. as described in co-owned U.S. Ser. No. 16/659,832 published as US20200289027A1 inter alia (the disclosure of which is hereby incorporated by reference herewithin in its entirety). Each stride (or cycle) is typically attached to or associated in memory with its estimated traveled distance (or stride length) and/or other gait measures corresponding to each stride, such as but not limited to all or any subset of the following: cadence, cadence variability, double support and single support of either leg, stride length asymmetry, and stride width.

Co-owned U.S. patent application Ser. No. 18/975,890 “Advanced Pedestrian Navigation Based on Inertial Gait Analysis and GPS Data”, incorporated by reference herein in its entirety, of which this application is a continuation-in-part, and/or U.S. patent application Ser. No. 17/500,744 published as US 2022/0111257, dated 13 Oct. 2021 and entitled “Efficient System Configured To Facilitate Physical Rehabilitation” also fully incorporated herein by reference, describe inter alia example method/s for partitioning motion, measured using an IMU, into intervals characterized or classified using types such as but not limited to an interval of “shake/s”, and other types of intervals such as “walk intervals”, and “Device Transition” referring to a change of the bodily position of the measurement device, e.g. from the user's pocket to her or his hand.

The system herein is typically configured to classify all data into five classes, and any data that is in “stride”, is again classified into activities, e.g., all or in a subset of the following activities which are all typically repetitive (walking, ascending, or climbing up stairs, descending or climbing down stairs, running, jumping, squatting, skipping, hopping, cycling). It is appreciated that being stationary, e.g., repeatedly doing nothing, either can or cannot be considered an activity.

The above data layer 30a is described in all or any subset of the following U.S. patent application Ser. Nos. 16/659,832, 17/500,744, US 2023/0137198 claiming priority from U.S. Ser. No. 63/272,839, the disclosures of which are hereby incorporated by reference in their entirety.

Operation 40. Standard mobility tests-use apparatus for collection of controlled measurement and a processor (on-device or remote through an Internet connection) to record or extract standard mobility test analysis e.g. timed up and go, sit to stand, static balance test/s, 6 minute walk test from the inertial data e.g. as described in any of the following co-owned US patent applications: Ser. No. 16/659,832 (US Publication US 2020/0289027), Ser. No. 17/500,744 (US Publication 2022/0111257), US 2023/0137198 which claims priority from U.S. Ser. No. 63/272,839 (the disclosures of which are hereby incorporated by reference herewithin in their entirety).

It is appreciated that mobility measures may be derived from measuring standard mobility tests, such as: TUG completion time, 180* turning time (left vs. right), sitting time, standing time, time in balance in the static balance test, magnitude and direction of movement and shakes during the static balance test, cadence and variability of sit-to-stand, etc.

Standard mobility tests in operation 40 may include all or any subset of:
40-1. TUG-www.physio-pedia.com/Timed_Up_and_Go_Test_(TUG)
40-2. Sit to stand-www.physio-pedia.com/30_Seconds_Sit_To_Stand_Test
40-3. 6-minute walk test www.physio-pedia.com/Six_Minute_Walk_Test_/_6_Minute_Walk_Test
40-4. Rhomberg balance test-www.physio-pedia.com/Romberg_Test
40-5. Four square step test-www.physio-pedia.com/Four_Square_Step_Test
40-6. The 4-Stage Balance Test-www.physio-pedia.com/The_4-Stage_Balance_Test

Or any other static balance test and analysis, such as but not limited to Sensory balance (dizziness-and-balance.com/testing/CDP/som_score.html), or sway analysis analysis (pubmed.ncbi.nlm.nih.gov/39520609/#:˜:text=Conclusions%3A%20The%20Brief%2DBESTest %20test,possible%20higher%20risk%20for%20falls.).

40-7. Reaction time in walking—the user is asked to walk back and forth and make U-turns (right or left) according to cues signaled by the device. The time between the cues' timestamps and the corresponding turns timestamps which may be extracted as described in co-owned US 2022/0111257 (the disclosure of which is hereby incorporated by reference herewithin in its entirety) is then averaged and evaluated as the user's reaction time.

The term outcome as used herein is intended to include any standard PRO aka Patient Reported Outcome or measurement or test or lab result e.g. cancer marker or clinician assessment or reported fall or even a binary observation such as “patient limps”, such as but not limited to those mentioned with reference to operations 40, 50.

Operation 50. Questionnaires and Patient Reported Outcomes-use apparatus for collection of questionnaires administered on a mobile app.

Questionnaires and patient-reported outcomes in operation 50 may include all or any subset of:
50-1. EQ-5D: A generic quality-of-life tool that can be adapted for use in chronic cancer patients to assess mobility, self-care, pain, usual activities, and mental health.
50-2. SF-36 or SF-12 (Short Form Health Surveys): General health measures to evaluate the broader impact of chronic cancer on quality of life.
50-3. Eastern Cooperative Oncology Group (ECOG) Performance Status: Rates a patient's ability to perform daily activities.
50-4. Karnofsky Performance Status (KPS): Measures functional impairment and prognosis.
50-5. EORTC QLQ-C30 (European Organization for Research and Treatment of Cancer Quality-of-Life Questionnaire): Widely used to assess health-related quality of life in cancer patients. It covers functional scales, symptom scales, and overall health status.
50-6. MD Anderson Symptom Inventory (MDASI): Measures the severity and impact of cancer-related symptoms on daily activities.
50-7. Brief Pain Inventory (BPI): Assesses pain severity and its effect on the patient's functioning.
50-8. Hospital Anxiety and Depression Scale (HADS): Screens for anxiety and depression levels in cancer patients.
50-9. Memorial Symptom Assessment Scale (MSAS): Evaluates a range of physical and psychological symptoms in cancer patients.
50-10. Cancer-Specific Measures: Bowel Cancer Screening Survey (BCSS) for colorectal cancer patients.
50-11. Prostate Cancer Quality of Life Instrument (PC-QoL) for those with prostate cancer.
50-12. Lung Cancer Symptom Scale (LCSS) tailored to the lung cancer population.
50-13. Functional Living Index-Cancer (FLIC): Measures daily functioning and the overall impact of cancer and its treatment.
50-14. Chemotherapy-Induced Symptom Assessment (CISA): Evaluates the side effects of chemotherapy on patients.

It is appreciated that standard outcomes e.g. those listed above with reference to operations 40, 50, may be administered less frequently or even not at all as the system described herein matures. for example, some or all of these standard outcomes may be used as a benchmark while the system is, in an earlier stage of maturity, still learning to derive, from IMU data, data which is just as good or almost as good as (directly measured) tools of operations 40, 50, without having to manually and/or in-clinic administer the tools of operations 40, 50. However, at a later stage the system may be mature enough to allow organizations (e.g. HMOs) to rely upon system results at which point standard tests may simply become superfluous other than, perhaps, for clinicians who were trained to use the standard outcomes listed above with reference to operations 40, 50 to guide clinical decisions. Thus as the system becomes mature, data generated from passive gait becomes more reliable hence typically more frequently used by the system's end-users, and direct measurement of standard outcomes e.g. those listed above with reference to operations 40, 50 may become less frequent.

Operation 60. Use dashboard and/or manage automatic reminders, to prompt the user regarding uncompleted data collection tasks (e.g. as per operations 40-50 above) and with regard to providing complementary data such as demographic information.

Operation 65. Use dashboard to interface with cooperating external sources to add information on the patient, such as the occurrence of clinical events e.g. falls, injuries, surgeries, and such as blood test results or other lab tests that indicate the presence or progression of cancer e.g. PSA, Complete Blood Count, or CA-125.

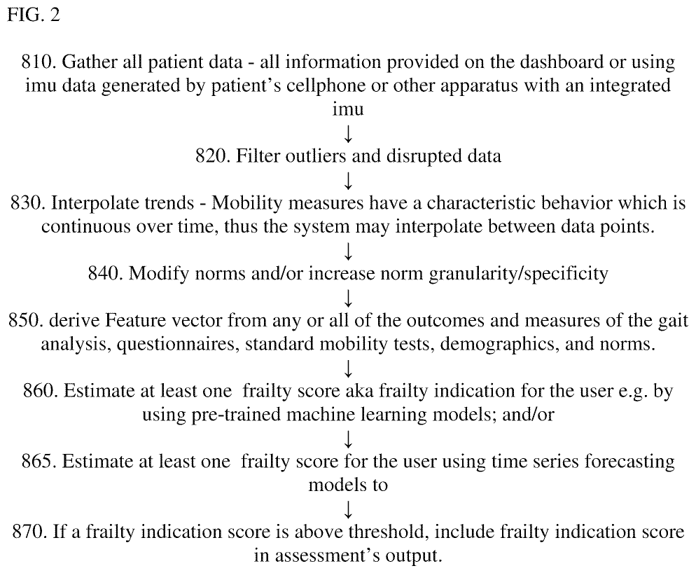

Operation 70. Every time a new data collection task is taken, e.g. each time the patient fills in the LCSS, or each time a passive walk is measured and uploaded, and/or when a new gait measurement is available (e.g. each time patient decides to walk, phone is carried on their body, and the app on the phone configured to perform methods herein wakes up to measure continuous IMU and other sensors data to and upload resulting measurements to the server). This typically becomes known to the system by triggers and/or upon a request made on the dashboard, an assessment procedure 80 is started/initiated which estimates current cancer indications, considering all longitudinal data, e.g. data being collected over time and or patient information, e.g. by performing all or any subset of the operations described below with reference to FIG. 2, in any suitable order, e.g. as illustrated.

The output of operation 70 typically determines when to initiate operation 80, and the results of operation 80 are typically used by operation 90. It is appreciated that the outputs of operations 820, 830, 840, and 860/865 may be combined in any suitable manner, e.g. as described herein in detail.

Operation 80: assessment e.g. as per FIG. 2.

Operation 90. Present the assessment output of operation 80 on the apparatus and dashboard, including all indications whose scores are higher than their corresponding thresholds. 100. On the dashboard, recommend doing any of the following: call patient, conduct in-person evaluation, change dosage or treatment.

It is appreciated that any output indication generating functionality described herein may communicate an output indication that has been generated to at least one entity (e.g. automatic SMS to HMO and/or a report document describing the deterioration which may be consumed via web dashboard or via API with EMR software platform such as Epic Systems or Cerner, or via email to an email address, stored in the system, of the entity e.g. HMO, carer, patient's family member, etc.).

The assessment procedure in operation 80 may include all or any subset of the operations 810, 820, . . . described below, in any suitable order e.g. as illustrated in FIG. 2.

Operation 810. Gather all patient data-all information provided on the dashboard or using the apparatus e.g. all or any subset of the following: demographic information, clinical conditions, treatment log, active and passive gait analysis, standard mobility test analysis, patient-reported outcomes and questionnaires, clinical events, injuries, surgeries, and medication. This is typically performed on the cloud, whereas all or any subset of operations 820-870 may be performed locally.

Operation 820. Filter outliers and disrupted data-sometimes, there might be some discrepancies in the estimated mobility measures e.g. gait analysis measures and mobility test measures. This is even more likely in passive gait measurements, since environmental effects are unknown. To reduce such noisy data, it is recommended to filter out (or at least indicate with lower certainty) strange data points. This task is possible when the patient's timeline is available, which enables the comparison of data points and the patient's reference. For example, a gait speed higher or lower by three standard deviations from the patient's weekly average gait speed may be considered an outlier. When many data points are available, as in passive gait monitoring, a daily or weekly (or any other timespan) aggregation, taking the median or the average, may be a solution to reduce outliers' noise.

Operation 830. Interpolate trends-Mobility measures (such as gait speed, stance asymmetry, ranges of motion, and PROs (patient-reported outcomes) typically have a characteristic behavior which is continuous over time, e.g. changes in these measures are not usually dramatic, and thus the system may interpolate between data points. For example, if an end-user has recovered from something, and his/her gait speed the previous week was 0.7, and this week is 0.9, then somewhere in between the gait speed may be assumed to have been 0.8 unless a dramatic event occurs (such as an injury). It is thus reasonable to interpolate some mobility measures between data points, as long as no medical incident has occurred, and produce trends for those measures. Linear interpolation between data points, e.g. of the data points collected in 810 (for each measure), and then filtered in 820, may be carried out, and then averaging the resulting interpolated data over a sliding window, to reduce measurement fluctuations afterward. The more frequent the measurements are, the more representative is the trend line. Gait active measurements may be taken between a few times a week and once every two weeks, depending on the natural progression of the patient's condition. The sliding window length may be determined accordingly to match the measurement frequency the patient is required in the program. For example, if a patient produces a few walks a day, the sliding window may be a day or two-day window, so that it covers several measurements. If a patient fills out one questionnaire a week, the sliding window's length may be 2 to 4 weeks.

Operation 840. Modify norms and/or increase norm granularity/specificity: norms are aggregations of patients' data and timelines over a specific population to compare to the patient's measures, indicating whether the patient is above or below the norms, for example, gait parameter norms by demographic data, such as normative values of gait speed by age and gender. Another example is the recovery pace in different parameters or outcome measures post-surgery, for instance benchmarks of 6-minute walk test distance by week for different surgeries and populations. Norms and benchmarks may be hardcoded, supported by literature, or computed by observations. Hence, when new data is collected and operation 80 is executed, there is an opportunity to regenerate norms. This process may alternatively run after a large set of new timelines and patient data is collected. Norms may become as granular and specific as available, e.g., gait norms for Parkinson's patients by gender, age, height, and weight.

Operation 850. Feature vector-a set of selected variables, e.g. the outputs of operations 810-840 composes a vector representing the patient's data at a specific timestamp (or date), in which every index allocates a specific variable. These variables are derived from any or all of the outcomes and measures of the gait analysis, questionnaires, standard mobility tests, demographics, and norms. One alternative is to define the variable value or to define a variable's value at a specific time-stamp on which the variable was not measured, to assign the corresponding measure's/variable's interpolated value at the specified timestamp. Another alternative is to set the value to be the actual value as last measured; typically the system may add a representation (e.g. number of days including fraction) of the time delta since this variable was last measured. The measurement's time may be embedded e.g. as described in arxiv.org/pdf/1907.05321. which describes providing a model-agnostic vector representation for time.

Operation 860. Estimate a frailty score aka frailty indication for the user e.g. by using pre-trained machine learning models. Any suitable method may be employed to train the models e.g. as described below. The models appropriate for operation 860 include regression models, such as but not limited to SVM, linear regression, neural network regression models, decision tree-based models, random forest, and XGBoost.

Operation 865. Alternative to operations 850 and 860, or in addition, and rather than describing the machine learning problem of generating a frailty score using a feature vector: vector -> score as a regression task where operation 860 herein performs regression analysis predicting a frailty score from the feature vector of operation 850, and regarding the frailty score prediction task as a time series forecasting that gets observations, each of which corresponds to a different timed measure or a static data point (gender, condition, etc.). In this case, every observation may include all or any subset of the time representation (0 when the variable has no timestamp, such as gender), the variable type representation, and the value representation, e.g. as described in arxiv.org/pdf/2109.12218. A model trained on this time series of observations is then applied to estimate the frailty indication score. The models appropriate for operation 865 include time series forecasting models such as but not limited to RNN, LSTM, and transformer-based models.

Operation 870. If a frailty indication score is higher than its threshold, include the frailty indication score in the assessment's output. Typically, if frailty is lower than threshold -> no indication, as described forthwith.

It is appreciated that cancer progression markers are relatively difficult and costly to obtain, and are hence infrequent, relative to the mobile phone-based frailty indication score very easily obtained, even for patients at home. It is appreciated that the frailty indication herein may be used to augment and or replace cancer progression markers as indicators of disease progression.

For example, for decision support or for automatic recommendation generation, frailty scores may be used to determine and/or justify whether to continue aggressive chemotherapy and/or radiotherapy as opposed to transitioning to end-of-life care or palliative care.

Predicting indications with clinical value may be provided, in accordance with certain embodiments.

Operations 860 and 865 refer to machine learning models that estimate the frailty indication score with clinical meaning, e.g. these models predict frailty from gait parameters. The models output scores that may correlate with standard clinically validated indexes, scores, tests, and outcome measures in general. To do so, these models are typically trained, using such outcomes (e.g. standard clinically validated indexes, scores, tests, for example, or any subset of those described herein with reference to operations 4D and 50 and 65) as labels. Training may, for example, include collecting and using all or any subset of EQ-5D, SF-36, or SF-12 and any specific chronic cancer PRO applicable for the patient's condition as labels. A regression model may be trained to output all or any subset of these scores. Deterioration in gait quality and/or decrease in gait speed and/or increased variability and/or dual-task cost measures may indicate an increase in the patient's frailty.

Regarding score thresholds, if any scores in the output subset have pathological or non-normal values, this indication may be included in the assessment output. Otherwise, e.g. if the estimated value is within the healthy range, no indication is included in the assessment output.

Training may occur before starting to use the system, and/or online or offline, during use of the system to yield new versions of the models which predict the clinical standard outcomes such as EQ-5D, SF-36, or SF-12 and any specific chronic cancer PRO e.g. like LCSS or FLIC or others from operation 50) more accurately. Typically, when training the model for the first use, the system, e.g. dashboard, is utilized for data collection only without providing any assessment output until enough data is collected. Any criterion may be used to enable the system to assess if “enough” data has been collected. Each model may have its own sample requirement of what constitutes being “enough”. For the simplest models (linear regression) that may be used as a starting point, at least 50 patients and 10 samples from each, including the whole range of clinical standard outcomes for any model and clinical standard outcome being estimated, may be deemed enough.

Referring again to operation 860, since not all data is required for the input of the models, the estimation of the frailty indication scores is performed with incomplete data. For operation 860, where a variable represents a measure that is missing, since it was never collected or collected a long time ago, the value provided for the feature vector may be null, and the models specified above are capable of handling null values.

Generally, the significance of such mobility-based indications is that they allow a much more continuous and/or much more unobtrusive and a far more cost-effective assessment of the patient's condition. Although the explicit outcome scores may be reported and collected through the system, they require the patient's adherence and engagement with the system, which, in reality, may be problematic, with respect to completing questionnaires and being asked sensitive questions. Thus replacing some or all requirements to fill out exhausting questionnaires, with measuring mobility tests using an app, enables monitoring the patient more frequently and/or remotely, and/or in a way which is less cumbersome for the patient. Even if there is a desire to exercise patience and continue with filling out questionnaires and being asked sensitive questions, the frequency of this may be reduced and perhaps may reach 0, as the system matures and gait parameters become more and more capable of replacing onerous self-report measures and/or cumbersome laboratory procedures, which require a patient to present herself or herself at a clinic, and are also less cost-effective.

For example, in operation 50 there may no longer be any daily or weekly need to have patients fill out questionnaires. Instead, an estimation of a patient's wellbeing, and hence an estimation of how disease is impacting the patient, may be derived from passive activity of the patient or simple walks, which are less onerous and/or more engaging than filling out exhausting questionnaires. Thus, measuring mobility tests, using an app, enables monitoring the patient more frequently and remotely and/or may serve as a screening process to determine which patient needs to be tested for clinical disease progression at a clinic.

Mobility measures may include gait parameters derived from the raw imu data such as cadence, stride length as well as parameters not pertaining to gait/walking such as TUG duration, sitting time, standing time, etc. indications of frailty and/or fatigue and/or imbalance may be derived from these mobility measure e.g. gait parameters.

According to embodiments herein, frailty is used as a general indicator of disease progression which may have a confidence level much lower than specific known disease markers derived clinically, such as cancer markers. However, frailty, as determined from phone data, thus essentially without cost, is cheaper and or more immediate and/or less subjective and or more frequently sampled and/or available much more widely, including for patients, who, for whatever reason, do not present themselves at a clinic and/or patients who are unable to cooperate with clinical procedures.

Oncologists may use this general indicator of disease progression to help guide treatment decisions, e.g. because indicators of disease progression indicate how well a patient is responding to treatment (chemotherapy, radiation, targeted therapy, etc.). If a treatment may not be working oncologists need to decide whether to adjust the therapy, e.g. by switching to different drugs, changing doses, or exploring alternative treatments like surgery or immunotherapy.

Alternatively, or in addition, after initial treatment, cancer patients are at risk of relapse (cancer returning). In this time period, patients may be less frequently monitored by their medical staff. Embodiments herein allow oncologists remote screening and general indicators of progression, which facilitate detection of signs of relapse as early as possible. Early detection allows for timely intervention, which may improve outcomes and provide more treatment options. Cancer patients have various characteristics and problems such as nausea pain control and constipation, plus undergoing radiation and or chemotherapy.

Known motor side effects include the following, all or any subset of which may be detected by analyzing IMU data provided by the cellphone of an end-user suffering from these side effects:

1. Tremors: Involuntary shaking or rhythmic movement, often affecting the hands, arms, or other body parts; Tremors may be used as a mobility measure; it is appreciated that tremors need not be measured directly; instead other mobility measures which are measured directly such as, say, cadence and and/or cadence variability may be used to predict such tremors using a suitable trained model. Labelled data to train the model may be obtained, say, by using technologies or apps such as Mon4t's neurological testing technology which delivers active and passive neurological monitoring. A Mon4t app may be integrated as a measurement/test in the app described herein and/or a clinician may document tremors during in-clinic visits; the system may then collect this data occasionally to use as labels, according to which the system may learn the relationship between e.g. gait mobility measures and tremors level.

It is appreciated that existence of tremors may be used as an indication of frailty (e.g. may be used to estimate frailty scores in addition to or alternatively to frailty estimation operations 860, 865 herein). For example, models may be trained to regress the level of tremors which may be measured continuously e.g. daily or hourly or every few minutes.

2. Bradykinesia: Slowness of movement, making everyday tasks more difficult or requiring more time to perform. It is appreciated that this may be detected by gait analysis deriving slow cadence and/or slow gait speed from IMU data of the end-user suffering from this side-effect.

3. Shuffling gait: for example, using a classifier similar to the 2-way classifier described in co-owned us 2020/0289027, entitled “assessment of A User's Gait” (the disclosure of which is hereby incorporated by reference herewithin in its entirety) where a new class (“shuffling”) is added to augment or replace the classes described in '027 (e.g. walking, stair-climbing, stair-descent, running etc).

4. other motor side effects affecting lower body such as freeze of gait, ataxia which may be detected in long walk tests e.g. the 6 (or 2) minute walk test when the patient is instructed to walk continuously without interruptions. The number and/or durations of stops may be counted.

A criterion for Freeze of gait may then be defined, say, as any stop (non-ambulation interval within a longer interval of ambulation e.g.) lasting more than half a second. The total freeze of gait duration ratio may be computed by summing durations of all such non-ambulation intervals and divided by the total measurement duration or the duration of the longer interval of ambulation.

It is appreciated that “Data layer 30a” described elsewhere herein typically includes motion semantic segmentation over time including partitioning motion into intervals which each belong to one of plural categories of recognized activity, including stops . . . .

A criterion for Ataxia (clumsiness) may then be defined e.g. as a function of the repetitive stride segments. A cosine similarity between each consecutive stride's imu representation may be computed. For example, if the percentage of different consecutive strides is, say, higher than 30%, then this percentage determines the estimated ataxia value. The threshold for different consecutive strides can be fit by validating that healthy people's ataxia scores do not exceed 30% when instructed to walk continuously without interruption, whereas patients with ataxia do usually get a score higher than 30%. Alternatively, ataxia can be measured using the consistency parameter that measures the deviation of the individual strides' imu representation e.g. compared to the mean repetitive imu representation, typically using methods described in co-owned U.S. patent application Ser. Nos. 16/659,832, 17/500,744, and US 2023/0137198 claiming priority from U.S. Ser. No. 63/272,839, the disclosures of which are hereby incorporated by reference in their entirety.

Motor side effects may cause tremors, stiffness, slowness of movement, or difficulty with coordination and balance.

Stiffness and slowness may be identified using gait analysis providing cadence (steps per minute) and gait speed, after first instructing the patient to move forward as fast as s/he is comfortably able to do. Alternatively or in addition, lower body Tremor may be detected. A cell app may ask patients or end-users to record themselves with their phone in their pocket while standing still for, say, 30 seconds. Compare magnitude of acceleration to baseline or norms; if a normal patient not suffering from lower body tremor is standing still, acceleration is near zero (not zero due to small-scale normal movements for balancing). If a person suffering from lower body tremor is standing still, her or his acceleration is higher (over-threshold e.g.

more than 2 std higher than the acceleration norm (new detection of lower body tremor) or if over 2 standard deviations from the person's baseline this may be considered a new deterioration even if that person is known to the system even previously as suffering from lower body tremor.

Typically the system is configured to derive at least one mobility measure for the static balance test e.g. duration in static balance, and/or magnitude of movement.

Upper body—system prompts the patient to hold the phone forward or in front of him/her, as far as possible from the body, the rest being the same as lower body measurement as described elsewhere herein.

Chemotherapy may cause motor side effects. Chemotherapy drugs may affect the nervous system, leading to various motor-related issues. The motor side effects may arise because several chemotherapy drugs, especially platinum-based drugs (such as cisplatin) and taxanes (such as paclitaxel), may damage the peripheral nerves, leading to peripheral neuropathy. This condition may interfere with fine motor skills and overall movement, and may result in all or any subset of tingling or numbness in the hands and feet and/or weakness and/or loss of coordination and/or balance problems and/or pain or sensitivity to touch e.g. when ambulating or any other difficulty in walking. It is appreciated that all or any of the above motor side effects may be detected using methods described elsewhere herein. Alternatively or in addition, classifier/s may be trained to detect gait patterns typical of sufferers from various neuropathic symptoms e.g. allodynia, muscle cramps, weakness, pain, numbness, weakness, balance difficulties or a dull, constant ache; these symptoms if reported may be used as labels to train such a classifier. For example, training data may include gait measurements from, say, 50-100 patients known to be suffering from diabetic neuropathy complication/s affecting gait, e.g. all or any subset of the above symptoms, and from, say, 100 healthy people. A classifier may then be trained to distinguish between gait patterns from these two groups.

A standard outcome of neuropathic-type pain e.g. burning, tingling, or stabbing (typically of a certain level e.g. a given score on visual analog scale for pain and in a certain part of the body such as in the feet, legs, or hands) may be estimated e.g. using any method described in co-owned U.S. application Ser. No. 19/049,400 entitled “System, Method and Computer Program Product for Monitoring Diabetics” and filed Feb. 10, 2025 (the disclosure of which is hereby incorporated by reference herewithin in its entirety). If some users in the system have actual clinical events of neuropathic-type pain which are already recorded in the system, these may be used as labels for training purposes.

Fall risk (or instability) assessment: measure a walk using a suitable app such as OneStep, testing e.g. TUG (timed up and go), sts (sit to stand).

Certain chemotherapy drugs, such as cytarabine and high-dose methotrexate, may affect the cerebellum, the part of the brain responsible for coordination and movement. Symptoms might include:
Ataxia (lack of muscle coordination, leading to unsteady movements)
Difficulty walking or maintaining balance
Tremors
Slurred speech

Chemotherapy may cause generalized fatigue, which may make it difficult for individuals to perform daily tasks or maintain normal muscle strength. This may affect mobility and fine motor control, especially when combined with other side effects like neuropathy.

Some chemotherapy treatments may contribute to muscle wasting or muscle weakness, making it harder to move or perform tasks that require strength and coordination.

Chemotherapy-induced Parkinsonism (symptoms similar to Parkinson's disease, such as tremors, rigidity, and slow movement) may occur due to the effects of chemotherapy on the central nervous system.

Cognitive impairments like memory problems, difficulty concentrating, and slowed thinking (sometimes referred to as “chemobrain”) may also indirectly affect motor function, particularly in tasks that require coordination between thinking and movement.

It is appreciated that difficulty walking (e.g. abnormality in one or more of: gait speed, cadence, stride-to-stride variability) and/or difficulty maintaining balance and/or tremors may be detected e.g. as described elsewhere herewithin. When using the 6 min walk test for example, indicative mobility measures may include distance walked and/or fatigue e.g. the second at which a significant (e.g. above-threshold) deterioration in gait speed occurs. When using, for example, the Static balance test, an indicative mobility measures may be duration in balance when movement <norm threshold.

Radiation therapy may cause motor side effects, which may lead to motor dysfunction. Common motor side effects of radiation include cerebellar damage and symptoms may include those described above as being associated with the cerebellum.

Radiation may cause neuropathy by damaging peripheral nerves, leading to peripheral neuropathy (similar to chemotherapy-induced neuropathy), for example when radiation is administered to the spinal cord, brain, or limbs. Symptoms include the following;
Numbness, tingling, or pain in the hands and feet
Weakness or difficulty coordinating movements, particularly in the extremities
Motor weakness or loss of fine motor skills
Radiation targeting the spinal cord or brain may result in radiation myelopathy, which may damage the nerve pathways responsible for motor function. Symptoms may include difficulty walking or maintaining balance which may be detected as described elsewhere herein and/or Hemiparesis (weakness on one side of the body) e.g. as evidenced by above-threshold (or above-baseline) asymmetrical gait mobility measures such as but not limited to stance asymmetry and/or step-length asymmetry.

It is possible for a course of radiation to be adjusted, e.g. dosage to be reduced, if a patient is found to be suffering from motor side effects. If a patient develops significant motor side effects, such as severe weakness, coordination problems, which may be identified by detecting ataxia, balance, tremors, and/or asymmetrical gai e.g. as described elsewhere herein and/or weakness in gait (e.g. below-threshold (and/or below-baseline) speed t.

Deterioration in quality of gait may indicate that radiation is affecting areas of the body or brain responsible for motor control, such as the spinal cord, brain, or nerves. If these side effects are severe and impair the patient's quality of life, doctors may reconsider the radiation plan. The medical team may choose to lower the radiation dose to prevent further harm to these structures. If, for example, new motor side effects during a course of radiation appear to suggest that the radiation is affecting the brain, spinal cord, or peripheral nerves, adjustments to the treatment plan for the ongoing course of radiation may be made to avoid permanent damage or worsen symptoms. This may involve reduced dosage and/or switching to a more targeted form of radiation, reducing the number of treatments, switching to an alternative therapy, altering the radiation schedule, combining radiation with other therapies (such as surgery, chemotherapy, or targeted therapy), or selecting a technique which is more precise in administering the required radiation. For example:
Intensity-Modulated Radiation Therapy (IMRT) may deliver a more focused dose to the tumor while sparing surrounding tissues.
Proton therapy may be used for its precision in delivering radiation to tumors with less collateral damage to nearby healthy tissue.
   Early signs of motor side effects may enable necessary adjustments to be made before the problem becomes severe. Regular neurological assessments may help detect these issues early but is costly, and takes a toll on the patient and on the medical team. In contrast, embodiments of the present invention may detect these issues without incurring these disadvantages.

The above side effects are also seen in individuals taking medications that affect the central nervous system, such as:
Antipsychotics (e.g., haloperidol, risperidone)
Antidepressants (e.g., selective serotonin reuptake inhibitors or SSRIs)
Parkinson's disease medications (e.g., levodopa)
Antiemetic drugs (e.g., metoclopramide)
   If motor side effects are severe, doctors may adjust the medication dosage, change the treatment, or recommend other therapies to manage or reduce the symptoms.

Cancer patients with dementia have a less favorable prognosis than cancer patients without dementia. Dementia may complicate both the treatment and overall management of cancer, which may lead to a number of challenges that affect survival rates, quality of life, and treatment options. Additionally, cancer patients with dementia may be less ideal candidates for surgery, if needed, inter alia this is because dementia may make it harder for patients to report side effects of treatment such as pain, fatigue, or other. This could delay the detection of complications or side effects, such as infections or progression of cancer, which may be managed more effectively with timely intervention. However, embodiments herewithin may substitute, for these patients, impaired or nonexistent ability to report side effects by extracting data suggestive of these side effects from the patients' phones accelerometer. Furthermore, frailty may make patients more susceptible to complications from cancer or its treatments, leading to unfavorable outcomes and decreased survival rates. Dementia often comes with frailty, and embodiments here within provide an ongoing indication of the current level of frailty to enable the patients' level of susceptibility to various treatments to be known on an essentially continuous basis.

It is appreciated that gait analysis, both momentary and over time, is described herein as being a useful indicator for cancer progression, however this is not intended to be limiting, and, more generally, gait analysis may be used as independent variables in machine learning new techniques for predicting progression of a wide variety of diseases, and conversely for identifying progress in convalescence from a wide variety of adverse medical episodes.

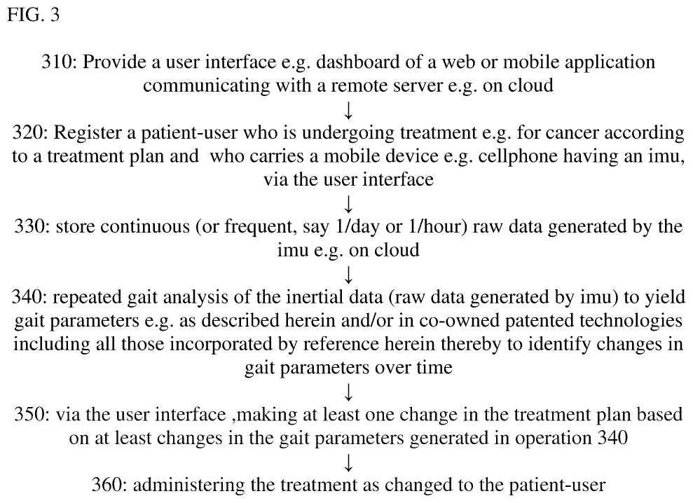

The system herein is useful when it is desired to treat patients with disease, by administering a treatment to a diseased patient who bears a mobile phone having an accelerometer according to a treatment plan; and using gait parameters derived from the accelerometer to monitor the patient including making at least one change in the treatment plan based on at least changes in the gait parameters, e.g. in accordance with FIG. 3 below.

It is appreciated that the system herein may be configured to predict a wide variety of standard assessments of clinical outcome e.g. self-report patient questionaires or PROs, clinician assessments, lab results e.g. cancer markers, disease-specific indexes, reported falls, etc.

Clinicians may change a patient's course of treatment (for cancer, diabetes, cognitive decline e.g.) based (in part) on results for outcomes such as EQ-5D, SF-36, SF-12, 6mwt and/or TUG. Given this, the system herein provides imu-based estimates of these results, without the cost of eliciting these outcomes from either the patient (for PRO's) or the clinician. For example, the system may be configured to predict 6mwt or TUG including its breakdown (sitting, standing, turning durations) without measuring all this directly.

While in future, physicians may be trained to make such decisions based directly on the imu data, in the meantime the system succeeds in effectively accommodating all md's as trained e.g. based on outcomes manually generated as is traditional (which the system herein may predict). Also, the system enables justification of recommendations and interpreting imu and mobility measures to yield indications of frailty, fatigue, and imbalance aids in making such decisions objectively and reliably.

It is anticipated that if the correlation of standard outcomes such as EQ-5D, SF-36 or SF-12, 6mwt and TUG with imu-generated predictions of these outcomes is considerably less than 100%, say 70%, a subset of md's and carers may prefer to continue requiring patients and/or clinicians to fill in questionnaires whereas other md's may prefer to use the system-generated predictions which are objective, passive, continuous, and almost cost-less. In the meantime, the system typically continues learning correlations between these standard outcomes and patient mobility measures.

It is appreciated that standard outcome estimates or predictions may be derived from gait analysis using any of the methods described in co-owned U.S. application Ser. No. 19/049,400 entitled “System, Method and Computer Program Product for Monitoring Diabetics” and filed Feb. 10, 2025 (the disclosure of which is hereby incorporated by reference herewithin in its entirety). For example, all or any subset of the following operations may be employed, where the patient data is as described herein, and the targets are any of the outcome measures mentioned herein by way of non-limiting example:
810. Gather all patient data within a suitable time-window e.g. all historical data in the system or only data for the last 1-2 year/s
820. Filter outliers and disrupted data from the data gathered in operation 810
830. Interpolate trends in the filtered data generated in operation 820
840. Modify norms and/or increase norm granularity/specificity
850. generate Feature vector/s representing patient/s status at a specific date
860. Estimate a standard outcome e.g. by using pre-trained machine learning models and/or using time series forecasting.

As more outcome measures are collected from the system's end-users, the system may improve its ability to generate predictions of these outcome measures at time-stamps in which directly measured outcomes are unavailable. According to certain embodiments, each time 10% more outcome measures have been collected or accumulated in the system, r models implementing frailty estimation operations 860 and/or 865 may be retrained to yield further system improvement. This improvement tends to move more clinicians out of non-adopting subset without requiring any clinician to abandon her or his traditional mode of working; any md or carer may still use questionnaires albeit, if and when s/he chooses, perhaps increasingly less frequently or, increasingly, just as additional confirmation.

References herein to cancer are merely exemplary and may instead be replaced, mutatis mutandis, with references to other diseases.

An advantage of certain embodiments is that the professionalism of modern medicine can paradoxically be the very factor which impedes its further progress. Specifically, despite a large body of knowledge accumulating in the field of gait analysis, gait analysis outputs are insufficiently used, to date, to guide treatment or interventions in the sense that these outputs could probably contribute far more than they are currently contributing in fact. One reason is that the very existence of standards of care which are not based on evidence accumulating in this relatively new field, may render any attempt to rely upon gait analysis outputs (which by definition involves departure from the current standards of care) unethical. Alternatively or in addition, because gait analysis is a relatively new field (often manned by researchers many of whom are not clinicians), gait parameters are inferior to known “standard outcomes” (which may be clinical/functional/quality of life measures including results of tools or scores administered/assigned in the clinic, PROs etc), in terms of ease of interpretation. Because clinicians are, by definition, unused to relying on gait analysis outputs, these outputs are, at this time, harder for clinicians to interpret.

Embodiments herein overcome these problems e.g. by providing a system which almost costlessly (no hardware, reliance on cellphones which patients use anyway) estimates or predicts whatever criteria (clinical test results, PROs, etc.) are currently used in evidence-based medicine to enhance ease of interpretation of an individual's gait analysis parameters. For example if TUG scores are conventionally used to determine whether or not a person can go out alone and/or whether or not a gait aid should be allocated to her/him, these scores can be estimated from the imu data generated by the cellphones of end-users of the system according to embodiments herein. Or, if a pre-diabetes diagnosis is made each time a patient presents with A1c between 5.7% and 6.4% and/or FPG between 100 and 125 mg/dL and/or OGTT between 140 and 199 mg/dL, and responsively, certain lifestyle changes such as diet modification and physical activity are recommended, or, if patients with A1c≥6.5% are assigned to, say, metformin or insulin, then A1c and/or FPG and/or OGTT scores can be estimated from the imu data generated by the cellphones of end-users of the system according to embodiments herein. Or, frailty may be estimated or predicted from gait parameters. Or, if “the 6MWT is used as a clinical basis for prescribing oxygen for home use” (pmc.ncbi.nlm.nih.gov/articles/PMC9095083/), 6 minute walk test scores can be estimated from the imu data generated by the cellphones of end-users of the system according to embodiments herein, and similarly any cancer markers or tests used to determine therapeutic decisions made in the care of cancer patients may be predicted or estimated.

Then, e.g. if treatments and their outcomes are fed back into the system, the system continues to learn to enable even better recommendations to be generated in future, e.g. as the system matures, by more direct reliance on gait parameters and fluctuation thereof over time e.g. as treatment proceeds, for example.

Inter alia, the respective confidence levels of system predictions of various standard outcomes or tests can be computed to enable clinicians to evaluate whether the system herein can currently be relied upon to drive decisions which rely upon these specific outcomes or tests and of course, as the system matures, these confidence levels may rise, enabling clinicians to adopt system recommendations selectably (to predict certain standard outcomes or tests but not others) in an evidence-based manner.

Yet another advantage of embodiments herein, is related to the fact that blood (or urine) tests are known, in modern evidence-based medicine, to be crucial far beyond their role in diagnosing actual diseases of the blood like leukemia. The role of blood tests in detecting, monitoring, and managing a wide array of non-blood-related conditions is a cornerstone of modern medicine; the versatility of blood tests is widely known allowing blood tests to serve as a powerful tool for doctors in diagnosing and managing a wide variety of diseases. In contrast, the role of gait analysis in detecting, monitoring, and managing a wide array of non-motor conditions such as cancer is far less accepted, if at all, in evidence-based medicine. The versatility of gait analysis may be equal to or surpass that of blood tests which would, in future, allow gait analysis, in future, to serve as a powerful tool for doctors in diagnosing and managing a wide variety of diseases, just as blood tests are today. Yet advancement toward that future can only occur if a system is proposed which: (a) allows the potential of gait analysis in, say, diagnosis and management of cancer to be learned; and/or (b) does not require risks to be taken (e.g. by abandoning recommendations generated the old way or by re-routing resources to expensive data collection, rather than these resources being allocated to other admittedly useful purposes as they are today) and/or (c) does not require burdensome data collection (burdensome for clinicians and/or for patients and/or in terms of cost borne by organizations), since currently, the advisability of reliance on gait analysis has yet to be proven scientifically. Embodiments herein achieve this.

It is appreciated that terminology such as “mandatory”, “required”, “need” and “must” refer to implementation choices made within the context of a particular implementation or application described herewithin for clarity, and are not intended to be limiting, since, in an alternative implementation, the same elements might be defined as not mandatory and not required. or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order, i.e., not necessarily as shown, including performing various operations in parallel or concurrently, rather than sequentially, as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g., in software, any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented, e.g., by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network—e.g., web-based system employing software, computers, routers, and telecommunications equipment, as appropriate.

Any suitable deployment may be employed to provide functionalities, e.g., software functionalities shown and described herein. For example, a server may store certain applications, for download to clients, which are executed at the client side, the server side serving only as a storehouse. Any or all functionalities, e.g., software functionalities shown and described herein, may be deployed in a cloud environment. Clients, e.g., mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis, e.g., triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware, or software module, which is known or which is described herein to have capabilities to perform a technical operation responsive to the state or condition. The technical operation may, for example, comprise changing the state or condition, or may more generally cause any outcome which is technically advantageous, given the state or condition or data, and/or may prevent at least one outcome which is disadvantageous, given the state or condition or data. Alternatively or in addition, an alert may be provided to an appropriate human operator or to an appropriate external system.

Features of the present invention, including operations which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation, and is not intended to be limiting.

Any suitable communication may be employed between separate units herein, e.g., wired data communication and/or in short-range radio communication with sensors such as cameras e.g., via Wifi, Bluetooth, or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network, or is tethered directly or indirectly/ultimately to such a node).

Any operation or characteristic described herein may be performed by another actor outside the scope of the patent application and the description is intended to include apparatus whether hardware, firmware or software which is configured to perform, enable, or facilitate that operation or to enable, facilitate, or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations, or functionalities described herein which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element, e.g., operation described herein may have all characteristics and attributes described or illustrated herein, or, according to other embodiments, may have any subset of the characteristics or attributes described herein.

It is appreciated that apps implementing any functionality herein may include a cell app, mobile app, computer app, or any other application software. Any application may be bundled with a computer and its system software, or published separately. The term “phone” and similar used herein is not intended to be limiting and may be replaced or augmented by any device having a processor, such as but not limited to a mobile telephone, or also set-top-box, TV, remote desktop computer, game console, tablet, mobile, e.g., laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node). Thus, the computing device may even be disconnected from e.g., WiFi, Bluetooth, etc., but may be tethered directly or ultimately to a networked device.

References herein to “said (or the) element x” having certain (e.g., functional or relational) limitations/characteristics, are not intended to imply that a single instance of element x is necessarily characterized by all the limitations/characteristics. Instead, “said (or the) element x” having certain (e.g. functional or relational) limitations/characteristics is intended to include both (a) an embodiment in which a single instance of element x is characterized by all of the limitations/characteristics and (b) embodiments in which plural instances of element x are provided, and each of the limitations/characteristics is satisfied by at least one instance of element x, but no single instance of element x satisfies all limitations/characteristics. For example, each time L limitations/characteristics are ascribed to “said” or “the” element X in the specification or claims (e.g. to “said processor” or “the processor”), this is intended to include an embodiment in which L instances of element X are provided, which respectively satisfy the L limitations/characteristics, each of the L instances of element X satisfying an individual one of the L limitations/characteristics. The plural instances of element x need not be identical. For example, if element x is a hardware processor, there may be different instances of x, each programmed for different functions and/or having different hardware configurations (e.g., there may be 3 instances of x: two Intel processors of different models, and one AMD processor).

1. A method for treating patients with disease, the method comprising
For at least one diseased patient who bears a mobile phone having an imu (built-in inertial measurement unit) and to whom treatment is being administered according to a treatment plan:
repeatedly deriving gait parameters from raw data generated by the imu, using a hardware processor, thereby to identify changes in the gait parameters over time; and
using the gait parameters, as repeatedly derived, to monitor the patient including making at least one change in the treatment plan based on at least the changes in the gait parameters.

2. A method according to claim 1, wherein the change in the treatment plan comprises transitioning to a new treatment protocol.

3. A method according to claim 1, wherein the change in the treatment plan comprises terminating a treatment protocol.

4. A method according to claim 1, wherein the treatment plan administers a dosage of a medical agent, and wherein the change in the treatment plan comprises changing the dosage.

5. A method according to claim 4, wherein the medical agent comprises a drug.

6. A method according to claim 1, wherein the medical agent comprises radiation.

7. A method according to claim 1 wherein the gait parameters are used to identify that the diseased patient is suffering from a known motor side effect of a treatment protocol being used and accordingly making said change in the treatment plan.

8. A method according to claim 1 wherein first and second treatment protocols are known to be characterized by first and second motor side effects, and wherein at least one gait parameter useful for recognizing the first motor side effect is computed in patients undergoing the first treatment protocol and typically not in at least one patient not undergoing the first treatment protocol, and wherein gait parameters useful for recognizing the second motor side effect are computed in patients undergoing the second treatment protocol and typically not in at least one patient not undergoing the second treatment protocol.

9. A method according to claim 1 wherein the treatment plan comprises a cancer treatment plan being administered to a cancer patient.

10. A system comprising at least one hardware processor configured to carry out a method for treating patients with disease, the method comprising:
Using the hardware processor, repeatedly deriving, for at least one diseased patient who bears a mobile phone having an imu (built-in inertial measurement unit) and to whom treatment is being administered according to a treatment plan, gait parameters from raw data generated by the imu thereby to identify changes in the gait parameters over time; and
using the gait parameters, as repeatedly derived, to monitor the patient including making at least one change in the treatment plan based on at least the changes in the gait parameters.

11. A computer program product, comprising a non-transitory tangible computer readable medium having computer readable program code embodied therein, said computer readable program code adapted to be executed to implement a method for treating patients with disease, the method comprising
For at least one diseased patient who bears a mobile phone having an imu (built-in inertial measurement unit) and to whom treatment is being administered according to a treatment plan:
repeatedly deriving gait parameters from raw data generated by the imu thereby to identify changes in the gait parameters over time; and
using the gait parameters, as repeatedly derived, to monitor the patient including making at least one change in the treatment plan based on at least the changes in the gait parameters.
